(19) United States
(12) Patent Application Publication
(10) Pub. No.: US 2005/0251006 A1
(43) Pub. Date: Nov. 10, 2005

(54) Method and system for remote post-processing of medical image information

(76) Inventor: Charles Joseph Dellis, Milwaukee, WI (US)

Correspondence Address: Dean D. Small;Armstrong Teasdale LLP, Suite 2600, One Metropolitan Square, St. Louis, MO, 63102, US

(21) Appl. No.: 10/825,470

(22) Filed: Apr. 15, 2004

(51) Int. Cl. A61B 5/05

(52) U.S. Cl. 600/407

(57) ABSTRACT

Methods and systems for performing medical diagnostic image processing are provided. The method includes configuring a medical imaging system for remote access to medical image information from a plurality of locations, with the medical image information stored within the medical imaging system. The method further includes performing post-processing operations on the medical image information from at least one of a plurality of locations, with the post-processing operations performed by individuals at the plurality of locations.

### BACKGROUND OF THE INVENTION

This invention relates generally to medical image post-processing and, more particularly, to performing medical image information post-processing at remote locations.

Patient diagnosis is a very important part of treatment procedures. The diagnostic procedures performed at medical imaging centers include CT scanning, MRI imaging, PET scanning, Nuclear Medicine, and X-ray imaging. The medical imaging exams received from these procedures typically have a large amount of data that provides information about a scanned body part. A medical imaging exam is an unprocessed medical image that is extracted while performing imaging operations. By applying post-processing (e.g., image processing) algorithms and processes, specific details about a body part or function, for example, can be extracted for interpretation. These specific details about a body part may enhance interpretation of the diagnostic results. However, post-processing is a computationally intensive process and normally needs a significant amount of processing time of the central processing unit of the device that performs the medical imaging, or a standalone workstation. In addition, the post-processing operation also typically requires the dedicated attention of the technologist who performs post-processing. This reduces the throughput of the scanning device and also reduces the number of patients being scanned because post-processing cannot be performed simultaneously with scanning.

In order to address these processing issues, medical imaging centers often save and transmit the medical imaging exams to a connected workstation or dedicated post-processing center to post-process them. However, the size of an average medical imaging exam (e.g., a plurality of medical images) is often in the order of several hundred megabytes, and sending and receiving such a large file becomes a cumbersome and time consuming process.

Further, known systems for post-processing do not provide dynamic access to the post-processing operations of medical images. These systems can only process and display the results after the medical images have been transmitted to the system. This results in a very time consuming and inefficient process.

Additionally, with increasing complexity in post-processing operations, post-processing has become an increasingly difficult task. In many post-processing centers, post-processing operations are performed by imaging operators or technologists having no specialized training in post-processing. The increasing complexity in post-processing makes it difficult for these individuals to perform the operations accurately. Moreover, errors at this step may result in downstream errors leading to an incorrect diagnosis (such as in image interpretation).

### BRIEF DESCRIPTION OF THE INVENTION

In one exemplary embodiment, a method for medical diagnostic image processing is provided. The method includes configuring a medical imaging system for remote access to medical image information from a plurality of locations, with the medical image information stored within the medical imaging system. The method further includes performing post-processing operations on the medical image information from at least one of a plurality of locations, with the post-processing operations performed by individuals at the plurality of locations.

In another exemplary embodiment, a medical imaging system is provided. The medical imaging system includes a medical imaging device for acquiring medical image information and a plurality of communication links between the medical imaging device and a plurality of remote locations, with the communication links configured for web-based communication. The medical imaging system also includes a user interface at each of the remote locations for accessing the medical image information within the medical imaging device using the plurality of communication links, and for post-processing the medical image information, with the post-processing performed by individuals at the remote locations using the medical imaging device.

### BRIEF DESCRIPTION OF THE DRAWINGS

FIG. 1 is a block diagram of a system enabling remote post-processing of medical images in accordance with an exemplary embodiment of the present invention.

FIG. 2 is a block diagram showing a medical imaging system in the system of FIG. 1.

FIG. 3 is a block diagram showing a workstation in accordance with an exemplary embodiment of the present invention that may be used in connection with the system in FIG. 1.

FIG. 4 is a block diagram showing a security layer in accordance with an exemplary embodiment of the present invention.

FIG. 5 is a block diagram showing a post-processing system in accordance with an exemplary embodiment of the present invention.

FIG. 6 is a block diagram showing a processing unit in accordance with an exemplary embodiment of the present invention.

FIG. 7 is a block diagram showing an operating system of a processing unit in accordance with an exemplary embodiment of the present invention.

FIG. 8 is a block diagram showing a TiP Virtual Assist (TVA™) platform in accordance with an exemplary embodiment of the present invention.

FIG. 9 is a block diagram showing a system on a TVA™ platform in accordance with an exemplary embodiment of the present invention.

FIGS. 10A and 10B are a flowchart showing the process of remote post-processing in accordance with an exemplary embodiment of the present invention.

### DETAILED DESCRIPTION OF THE INVENTION

Various embodiments of the present invention provide for remote post-processing of medical image information. Thus, post-processing may include any processing after acquiring medical images as is known, including, but not limited to performing manipulation, enhancement, classification, and rectification operations on a digital medical image. Further, the medical image may be, for example, a Computer Tomography (CT), Medical Resonance Imaging (MRI), Nuclear Medicine, PET, Ultrasound, and/or an X-ray image.

Various embodiments of the present invention provide methods and systems for remote post-processing of medical image information. In accordance with various embodiments of the present invention, post-processing operations are performed remotely by an individual and/or an entity that, for example, has expertise in post-processing. In the various embodiments, the individual and/or entity may or may not be related to the medical imaging center where the medical image information, including the medical images are acquired by the medical imaging system. In various embodiments, post-processing operations, therefore, may be performed outside the premises of the medical imaging center. In various embodiments, the entity may enter a contractual agreement with the medical imaging center and provide, for example, on-demand post-processing operations to the medical imaging center. The entity may contractually employ technologists that perform post-processing operations remotely for the medical imaging center. The technologists perform post-processing operations remotely by accessing the secure network of the medical imaging center, for example, via a public telecommunication network as described herein. Further, the individual(s) performing the post-processing may have specialized expertise in specific areas of post-processing, such as 3D post-processing, cardiac function analysis, advanced vessel analysis, virtual colonoscopy, cross-modality image fusion, and the like.

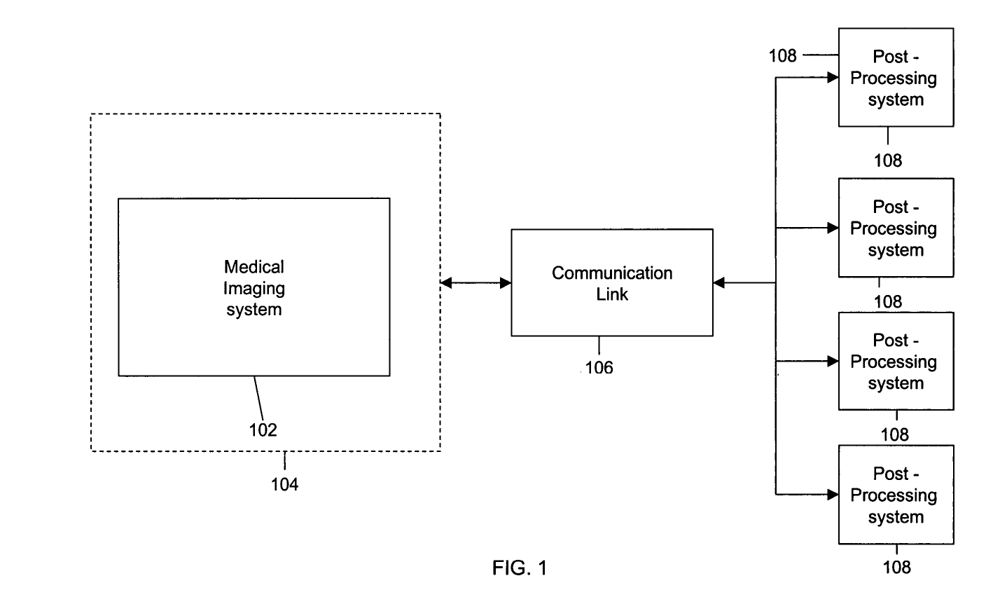

FIG. 1 shows a system 100 enabling remote post-processing of medical images in accordance with various embodiments of the present invention. The system 100 includes a medical imaging system 102, a security layer 104, a communication link 106 (e.g., a public telecommunication network), and one or more post-processing systems 108.

Medical imaging system 102 is connected to post-processing system 108, which may be an entity performing post-processing remotely. Medical imaging system 102 may be an independent diagnostic unit performing operations including, for example, CT scan, X-ray, PET scan, MRI, etc. In various embodiments of the present invention, medical imaging system 102 may be a department in a healthcare unit. The healthcare unit may perform one or more of the abovementioned operations for diagnosis, surgery and medicinal treatment.

In various embodiments of the present invention security layer 104 links medical imaging system 102 to, for example, a public communication network. However, depending on the type of communication link 106 or communication requirement, security layer 104 may be removed. Security layer 104 encrypts requests for medical imaging information and provides for communicating encrypted information to post-processing system 108 via communication link 106. An example of communication link 106 is public communication network, such as the Internet. In one embodiment, the connection between medical imaging system 102 and post-processing system 108 is provided by a broadband Internet connection. Thus, communication link 106 may communicate encrypted information through a secure data transfer tunnel. A tunnel is generally a link that uses a data encapsulation protocol that encapsulates the encrypted data in a manner that the data, when received by communication link devices, such as routers, appears like other unsecured data that is transferred over the communication link. The tunneling may be enabled using tunneling protocols such as, for example, Point-to-Point Tunneling Protocol (PPTP), Layer Two Tunneling Protocol (L2TP), and the like. In various embodiments, post-processing system 108, after receiving the request for post-processing, securely accesses medical imaging system 102 and performs post-processing remotely on medical imaging system 102.

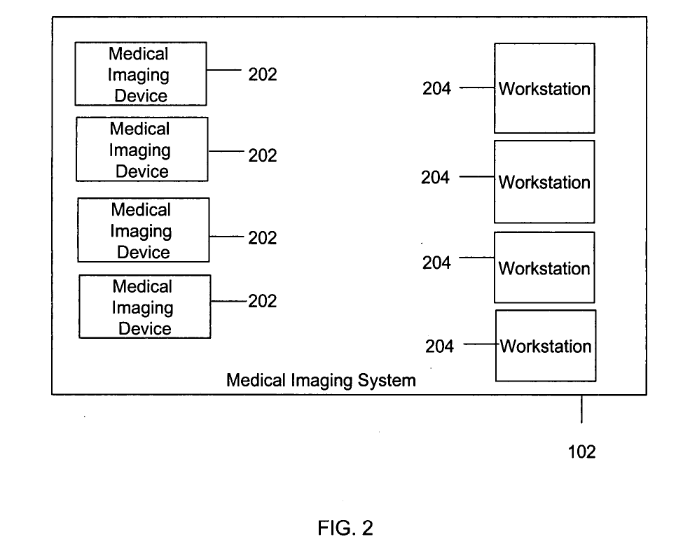

FIG. 2 is a block diagram of medical imaging system 102. As shown, medical imaging system 102 includes one or more medical imaging devices 202 and one or more workstations 204. Medical imaging device 202 performs scanning operations such as CT scans, X-rays, PET scans, MRI, etc., and stores the corresponding medical image information, including medical images within its memory (not shown), for example, a local drive, or on one or more workstations 204. Workstation 204 may be an internal or an external unit to medical imaging device 202. In various embodiments of the present invention, the medical images may be remotely accessed from medical imaging device 202 or from workstation 204. Also, the post-processing of these medical images may be performed on medical imaging device 202 or workstation 204.

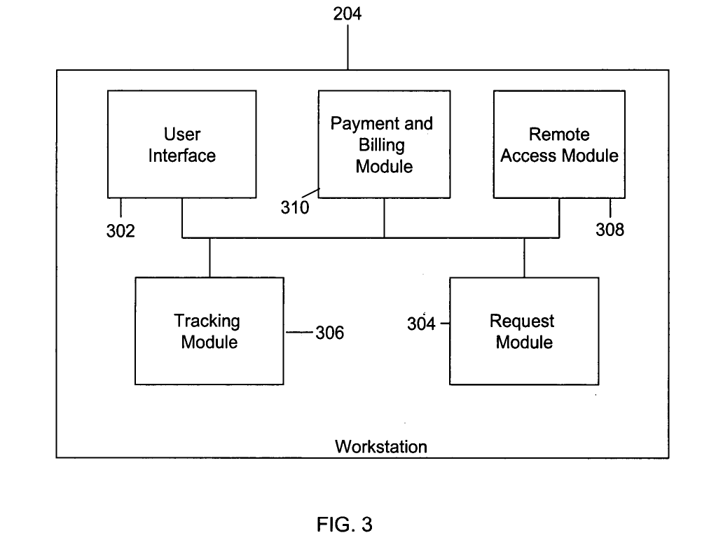

FIG. 3 is a more detailed block diagram of workstation 204. These sub-components of workstation 204, as shown in FIG. 3 may be used for facilitating post-processing. As shown, workstation 204 includes a user interface 302, a request module 304, a tracking module 306, a remote access module 308, and a payment and billing module 310. User interface 302 supports un-processed and post-processed formats of medical imaging files. For example, user interface 302 recognizes image file formats such as ‘.img’ and ‘.hdr’. User interface 302 further provides co-browsing functionalities. In various embodiments of the present invention, the co-browsing functionalities may be enabled using, for example, a server, such as a server operating using a base code from an open source Virtual Network Computing (VNC) server, for example, an x0rfbserver. A VNC server facilitates remote browsing and viewing. In various embodiments, this server may use an X Server on workstation 204 to obtain the frame buffer contents and analyze the screen for changes. In various embodiments of the present invention, interactions (e.g., between client programs and user inputs) are managed by tracking the changes on user interface 302 and refreshing the remote system viewer. The X Server communicates changes in the frame buffer to a remote viewer (in post-processing system 108). In various embodiments of the present invention, the changes may be specified using a Remote Frame Buffer (RFB) protocol. In addition, the open source VNC server may include a ‘gemsvnc’ server that implements a command processing system that interprets commands to guard or block certain areas of the screen on the remote viewer. The open source VNC server, in its various embodiments, also supports an ‘http’ based connection from a java enabled browser and automatically downloads an applet based viewer to connect to workstation 204 from post-processing system 108.

Request module 304 provides an interface for receiving custom or user defined requests for post-processing. The requests received by request module 304 may be defined, for example, by one or more of the following parameters:

1. The body part or organ to be post-processed: The post-processing request may be different, for example, for an artery and a bone marrow. Different post-processing algorithms may need to be applied to extract features or requested information from the respective body parts.

2. Response time: The criticality of the post-processing may determine, for example, the response time. Request module 304 receives response time values that may vary, for example, from thirty minutes to several days. These response times may be predetermined, for example, based upon the type of medical images, contractual obligations, and the post-processing procedures to be performed, among others.

3. Type of post-processing: Request module 304 further receives inputs to select between, for example, different types of processing, such as 3D post-processing, cardiac function analysis, advanced vessel analysis, virtual colonoscopy and cross-modality image fusion.

These parameters facilitate efficiently presenting the request for post-processing at post-processing system 108.

Tracking module 306 monitors central processing unit (CPU) usage including internal scanning processes and external post-processing operations. Tracking module 306 also monitors the time for which post-processing operations are performed on medical imaging system 102 by post-processing system 108. For example, during the daytime, a simultaneous high rush for scanning (e.g., high scanning use) and post-processing operations may result in faults or errors in medical imaging. Thus, medical imaging system 102 may become slow in scanning. In one exemplary embodiment, tracking module 306 monitors such activities and sends notification to remote access module 308, which adjusts the CPU time allocated to the remote post-processing operation to provide uninterrupted functioning of imaging operations.

Remote access module 308 controls access to medical image information to be post-processed. Remote access module 308 may receive access commands or information manually or automatically. For example, the operator of medical imaging device 202 may provide instructions to deny any remote access to medical imaging system 102 during a certain time period. Remote access module 308 then blocks remote access during that time period. Remote access module 308 further receives CPU usage information from tracking module 306 to control access to post-processing system 108 for post-processing. For example, if the medical imaging device 202 is heavily loaded with scanning operations, tracking module 306 generates information regarding high CPU usage, and based on this information, remote access module 308 may place any remote access to medical imaging system 102 for post-processing at a low priority. This ensures scanning at the medical imaging system 102 with less errors and more throughput. Further, remote access module 308 may provide notification when remote access is established. Thereafter, a user at the medical imaging system 102 may terminate the access as desired or needed.

Payment and billing module 310 provides payment and billing information to medical imaging system 102. Payment and billing module 310 facilitates on-line contractual payment to post-processing system 108.

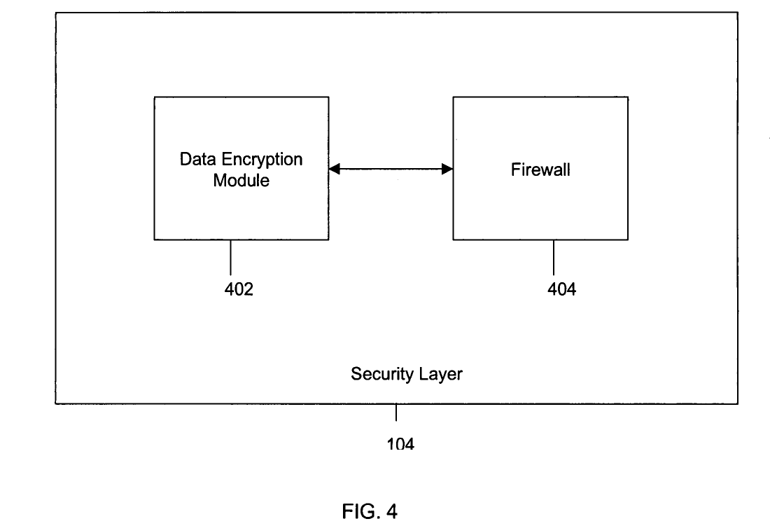

FIG. 4 further shows the components of security layer 104. In various embodiments of the present invention, security layer 104 may be enabled, for example, using a Virtual Private Network (VPN). Security layer 104 includes a data encryption module 402 and a firewall 404. Data encryption module 404 encrypts the post-processing information generated by request module 304. Data encryption module 402 may perform encryption using, for example, symmetric or non-symmetric data encryption techniques. Examples of data encryption algorithms used in a VPN include Triple-DES, AES and IDEA. Firewall 404 provided by security layer 104 secures access to the post-processing information in medical imaging system 102. The firewall architectures may be selected, for example, from one of the following architectures: Stateful Inspection, Proxy based, and Packet Filtering. It should be noted that the examples of data encryption module 402 and firewall 404 are merely exemplary, and different or additional components may be provided as desired or needed.

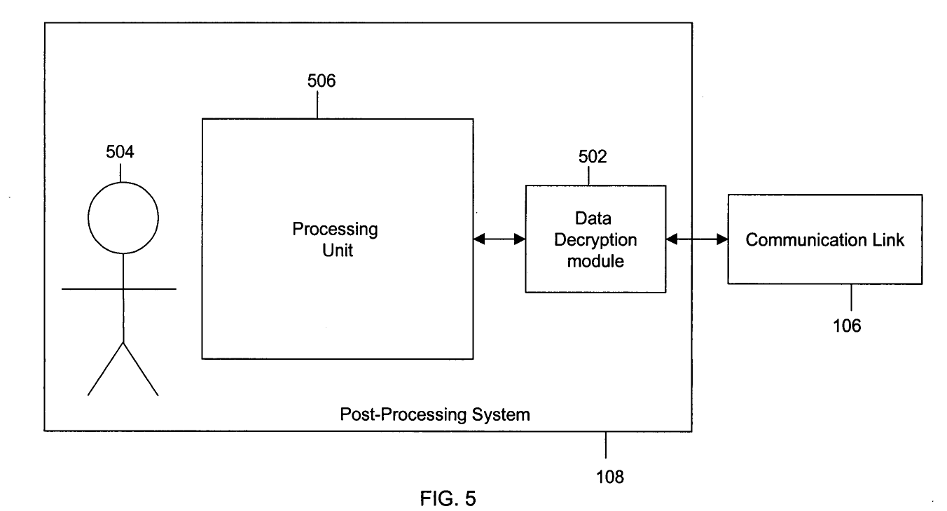

FIG. 5 is a more detailed block diagram of post-processing system 108. Post-processing system 108 includes a data decryption module 502, a technologist 504 and a processing unit 506. Data decryption module 502 decrypts the encrypted post-processing information received from communication link 106. One or more technologists 504 remotely perform post-processing operations by remotely accessing medical imaging device 202 or workstation 204 via communication link 106. Technologist 504 remotely logs onto secure layer 104 of medical imaging system 102 via processing unit 506, and receives the request(s) for post-processing. The request(s) for post-processing may include specifications relating to the type of post-processing, the body part to be post-processed, and the like.

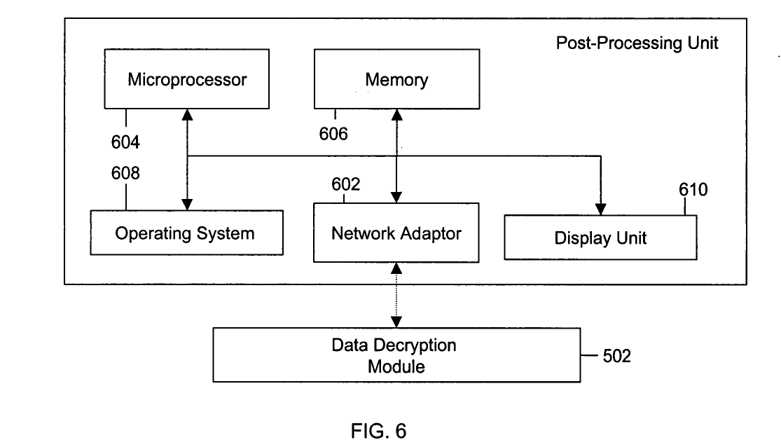

FIG. 6 is a more detailed block diagram of processing unit 506. Processing unit 506 includes a network adaptor 602, a processing unit, such as a microprocessor 604, a memory 606, an operating system 608 and a display unit 610. Network adaptor 602 communicates with data decryption module 502 and receives the decrypted post-processing information. Microprocessor 604 processes requests communicated to processing unit 506. Microprocessor 604 uses memory 606 while processing the request. Operating system 608 provides technologist 504 an interface for remotely accessing the request and performing post-processing operations on medical imaging system 102 from a remote location. Information (e.g., medical images) is displayed on display unit 610 and a user input (e.g., keyboard) may be provided to control processing unit 506.

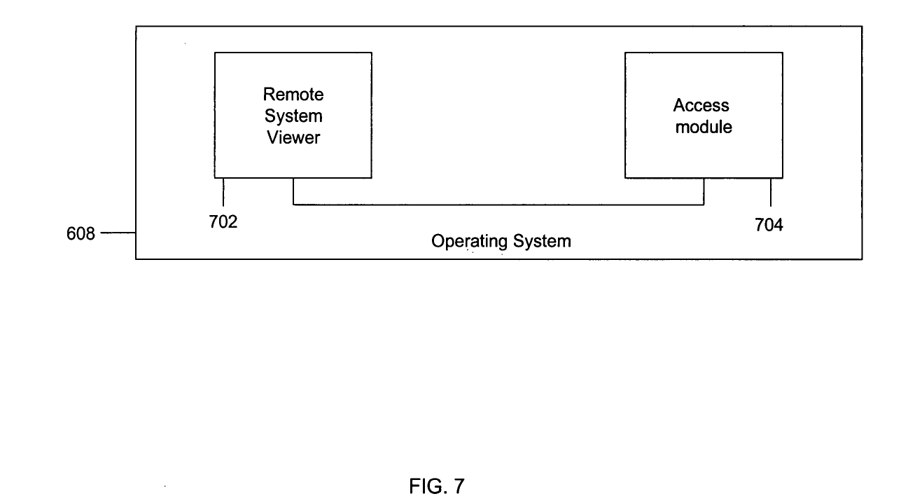

FIG. 7 is a block diagram of operating system 608. Operating system 608 includes a remote system viewer 702, and a secure access module 704. Remote system viewer 702 provides a user with different functionality as described herein, such as, for example, viewing and co-browsing. In various embodiments, the co-browsing functionality of remote system viewer 702 enables access to user interface 302 of workstation 204 by technologist 504 (shown in FIG. 3). Remote system viewer 702 may be, for example, a standalone VNC viewer application, or a java enabled browser accessing the VNC server in user interface 302 and automatically downloading the applet version of the VNC viewer. Secure access module 704 enables secure access, for example, to the private network of medical imaging system 102. Further, technologist 504 may be required to enter specific information, such as a user name and password, to access the medical image information, which information is confirmed by the remote access module 308 and an authentication server to allow access.

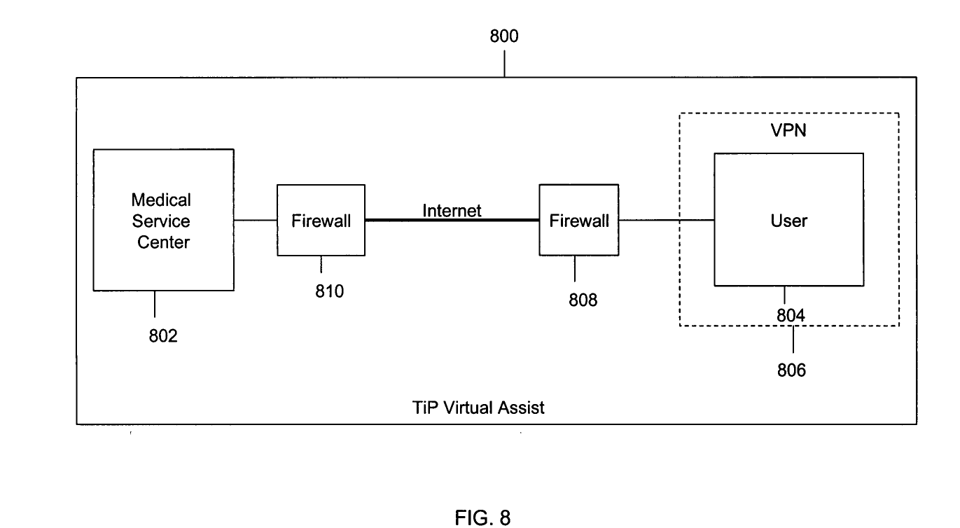

Various embodiments of the present invention may be implemented, for example, using a remote communication application, such as a TiP Virtual Assist (TVA™) platform available from GE Medical Systems. The TVA™ platform is shown in FIG. 8. As shown, TVA™ 800 platform connects a medical service center 802 to, for example, a user 804 through a Virtual Private Network 806. VPN 806 includes a firewall 808 at medical service center 802 side and a firewall 810 at the user 804 side. TVA™ 800 platform uses a high-speed broadband connection to access, for example, medical image information or medical training information stored within medical imaging devices and/or at the medical service center 802. To facilitate interaction, for example, between a system at medical service center 802 and, for example, user 804, TVA™ 800 platform provides co-browsing applications using a Remote Console Observation and Control (RCOC) module. The RCOC module uses a VNC server for facilitating remote access of user interface by user 804. TVA™ 800 platform may also include a telephone connection that links, for example, user 804 directly to medical service center 802.

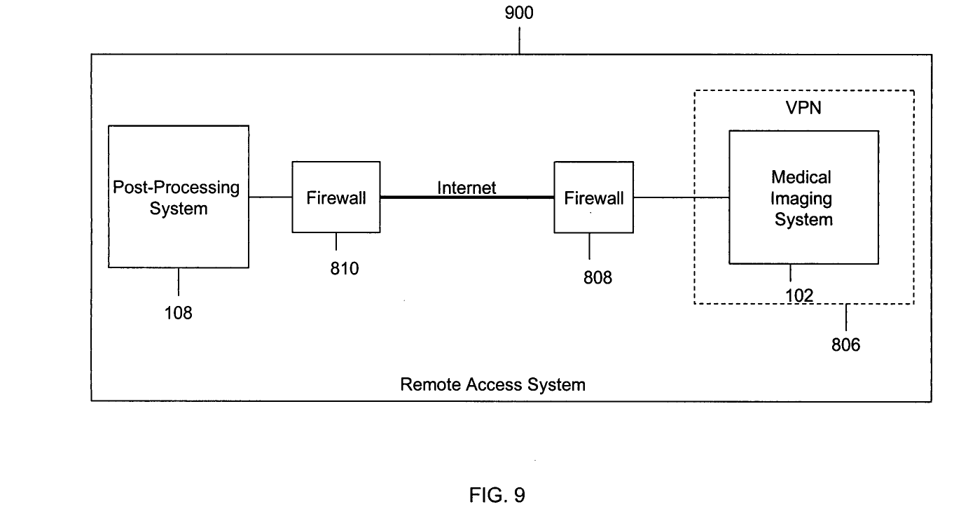

FIG. 9 shows a remote access system 900 constructed to provide post-processing operations as described in more detail herein. Such a remote access system 900 may be provided, for example, using a TVA™ 800 platform. However, it should be noted, any system capable of providing remote access as described herein may be implemented. As shown, remote access system 900 is used by medical imaging system 102 to send post-processing request(s) to post-processing system 108. Access to medical imaging system 102 and post-processing system 108 is restricted by firewall 810 and firewall 808. Remote access system 900 also enables co-browsing, for example, through a Remote Console Observation and Control (RCOC) module.

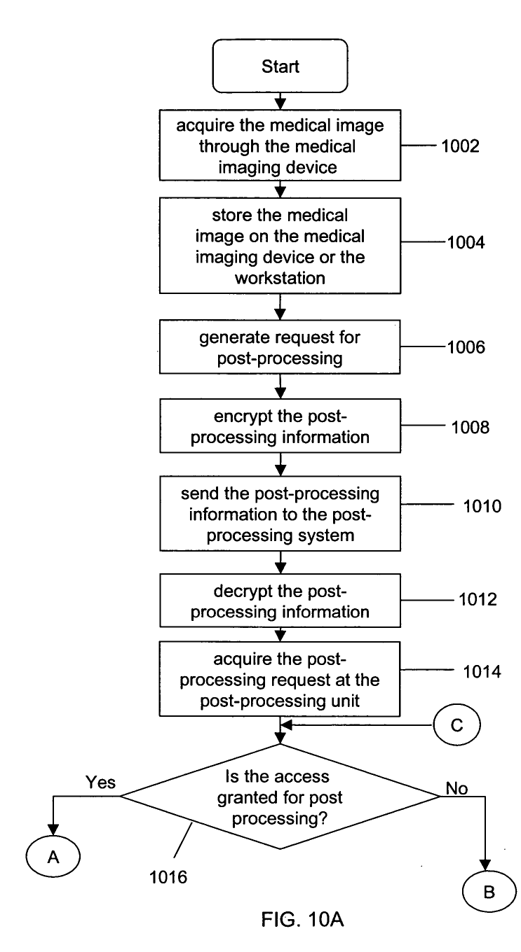

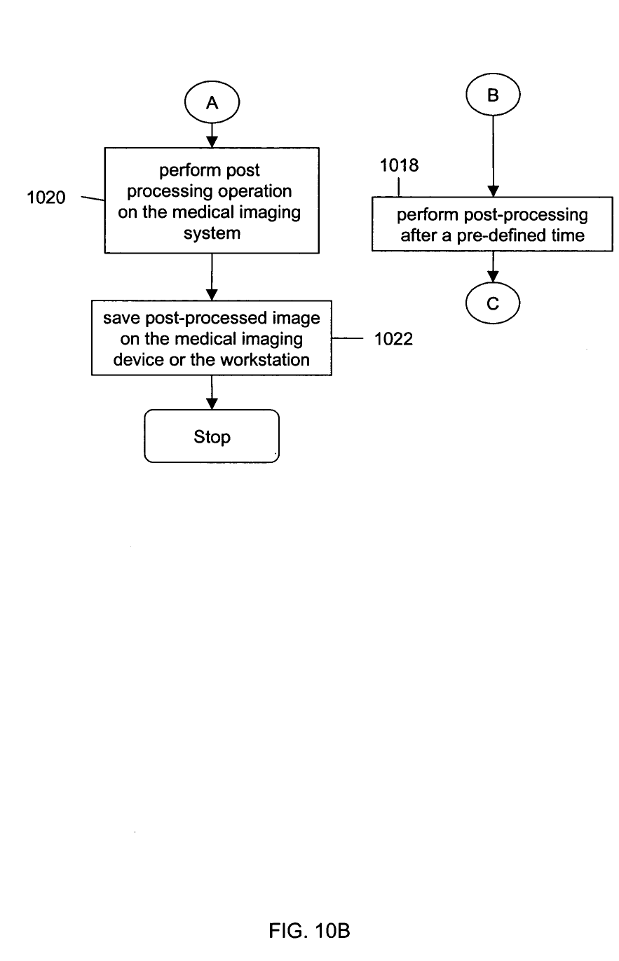

FIG. 10A and FIG. 10B are a flowchart for remotely performing post-processing in accordance with various embodiments of the present invention. At 1002, medical image information is acquired, and may include, for example, medical imaging device 202 (shown in FIG. 2) acquiring images of a patient. The images may be, for example, a Computer Tomography (CT), Medical Resonance Imaging (MRI), Ultrasound, or an X-ray image. The acquired image is stored in medical imaging system 102 (shown in FIG. 2), at 1004. This may include, for example, storing acquired images in medical imaging device 202 and/or workstation 204 (shown in FIG. 2). At 1006, request module 304 (shown in FIG. 3) in medical imaging system 102 generates a request for post-processing. The request for post-processing may be generated manually by the imaging operator performing imaging, or by a physician at the medical imaging system 102, or automatically, for example, upon completion of a scan. As described herein, the request for post-processing may include specifications such as, the type of post-processing, response time, and the like. Based on these specifications, post-processing is performed at medical imaging system 102 as described herein.

At 1008, the request for post-processing is encrypted by encryption module 402 (shown in FIG. 4). The post-processing request is encrypted in order to ensure secure transfer over communication link 106. At 1010, the request for post-processing is communicated over communication link 106 through, for example, secure Internet tunnels. At 1012, the encrypted post-processing information (e.g., post-processing request) is received at post-processing system 108 (shown in FIG. 1), and is decrypted by decryption module 502 (shown in FIG. 5) of post-processing system 108 (shown in FIG. 5). At 1014, the decrypted information is acquired by processing unit 506 (shown in FIG. 5) of post-processing system 108. At 1016, technologist 504 (shown in FIG. 5) accesses medical imaging system 102 via secure access module 704 (shown in FIG. 7). Secure access module 704 enables secure access to the medical images at medical imaging system 102. The request for access is sent by secure access module 704 to remote access module 308 (shown in FIG. 3).

Remote access module 308 may allow secure access based on, for example, inputs received from the imaging operator, the physician, or may use information generated by tracking module 306 (shown in FIG. 3). The imaging operator or the physician may deny remote access to medical imaging system 102 based on the conditions locally at medical imaging system 102. For example, in the case of repair of the medical imaging device 202 or workstation 204 (shown in FIG. 2), remote access may be disabled. Access to medical imaging system 102 also may be disabled based on the information received from tracking module 306. The information generated by tracking module 306 may include, for example, CPU utilization information. If, for example, the CPU utilization information indicates that the usage is above a threshold value, remote access module 308 may automatically deny access. For example, access may be denied when a large number of imaging operations are being performed in medical imaging system 102.

At 1016 if access is denied, then at 1018, technologist 504 is notified (e.g., notification provided on display unit 610 (shown in FIG. 6)) that access has been denied. Thereafter, for example, after a predetermined time period, at a pre-defined time, or after a determination is made that access is available, a subsequent notification is provided and access provided. The predetermined time period may vary based on information provided by remote access unit 308 to secure access module 704. For example, if medical imaging system 102 sends information that a high volume of traffic for imaging is being received, the pre-defined time may be a suitable time in the evening, when the imaging operations are complete at medical imaging system 102. At 1016, if access is granted, then at step 1020, the processing unit 506, remotely accesses the medical images at medical imaging system 102 and post-processing on the medical imaging device 202 and/or workstation 204 may be performed. The post-processed information is then saved on medical imaging device 202 or workstation 204 at 1022. The post-processed images are later used, for example, by the imaging operator or the physician at medical imaging system 102 for diagnosis.

Various embodiments of the present invention allow for performing post-processing remotely at a post-processing system, using medical image information stored on a medical imaging system. Performing post-processing remotely reduces the load of post-processing for the medical imaging center. By performing post-processing remotely, the time for post-processing is reduced from the medical imaging system processing system, which increases imaging throughput and further ensures proper operation of the medical imaging device during heavy imaging traffic times.

Further, various embodiments of the present invention provide a medical imaging center with high quality post-processing results. Using, for example, an entity that has an expertise in post-processing increases the quality of post-processing operations. A pool or group of post-processing specialists may be provided. Human errors in post-processing of medical images also may be reduced.

Further, various embodiments of the present invention perform post-processing at the medical imaging system. An average medical imaging exam typically has a size in the range of several hundred megabytes, and sending an exam of that size over, for example, the public telecommunication network may result in a post-processing cycle of days. Various embodiments of the present invention are configured to transmit a request for post-processing to the technologist at the post-processing system, without the need to send, for example, any medical images. By remotely accessing the medical imaging center and performing the post-processing operations without transferring the data, the post-processing process is improved.

Further, various embodiments of the present invention allow real-time post-processing using different remote access platforms, such as, for example, the TVA™ platform. The co-browsing functionalities provided by the RCOC module along with the direct telecommunication link provided between the systems provides improved access and interaction between the technologist performing post-processing and the medical imaging center.

Further, various embodiments of the present invention provide remote interpretation of the post-processed image by remotely consulting an expert at the processing unit. Thus, the medical imaging center also can obtain expert guidance for certain critical cases that cannot be handled by certain physicians. Further, medical imaging centers can optimize workflow by selecting a post-processing service based upon, for example, the type of processing to be performed.

A technical effect of various embodiments of the present invention is to provide more efficient post-processing operations. In various embodiments, the post processing is performed remotely by a technologist having an expertise in a particular type of post-processing. Another technical effect of various embodiments of the present invention is to track the post-processing operation on a runtime basis. Yet another technical effect of various embodiments of the present invention is to enable performing post-processing remotely using information at the medical imaging center. In various embodiments, the large volume of data linked to a medical imaging exam is not transferred and the post processing is performed by remotely accessing this data.

Various embodiments of the present invention may be implemented or embodied in the form of a computer system. Examples of a computer system include a general-purpose computer, a programmed microprocessor, a micro-controller, a peripheral integrated circuit element, and other devices or arrangements of devices that are capable of implementing the various embodiments of the present invention.

The computer system may include a computer, an input device, a display unit and an interface, for example, for accessing the Internet. The computer may include a microprocessor. The microprocessor may be connected to a communication bus. The computer may also include a memory. The memory may include Random Access Memory (RAM) and Read Only Memory (ROM). The computer system further may include a storage device, which may be a hard disk drive or a removable storage drive such as a floppy disk drive, optical disk drive, and the like. The storage device can also be other similar means for loading computer programs or other instructions into the computer system.

The computer system executes a set of instructions that are stored in one or more storage elements, in order to process input data. The storage elements may also hold data or other information as desired or needed. The storage element may be in the form of an information source or a physical memory element within the processing machine.

The set of instructions may include various commands that instruct the processing machine to perform specific operations such as the processes of the various embodiments of the present invention. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

While the invention has been described in terms of various specific embodiments, those skilled in the art will recognize that the invention can be practiced with modification within the spirit and scope of the claims.

1. A method for medical diagnostic image processing, said method comprising:
configuring a medical imaging system for remote access to medical image information from a plurality of locations, the medical image information stored within the medical imaging system; and
performing post-processing operations on the medical image information from at least one of the plurality of locations, the post-processing operations performed by individuals at the plurality of locations.

2. A method in accordance with claim 1 wherein the individuals comprise persons having expertise in different areas of medical imaging.

3. A method in accordance with claim 1 wherein the remote access comprises access via a public telecommunication infrastructure.

4. A method in accordance with claim 1 wherein the remote access comprises access via at least one virtual private network.

5. A method in accordance with claim 1 wherein the medical imaging system is located at one physical location and the plurality of individuals are located at different physical locations.

6. A method in accordance with claim 5 wherein the medical imaging system comprises at least one medical imaging device and at least one post-processing workstation.

7. A method in accordance with claim 5 wherein the individuals are not associated with the physical location having the medical imaging device.

8. A method in accordance with claim 1 further comprising accessing the medical image information directly from the medical imaging system.

9. A method in accordance with claim 8 wherein the medical image information is accessed in real-time.

10. A method in accordance with claim 1 further comprising post-processing the medical image information using the medical imaging system.

11. A method in accordance with claim 1 further comprising receiving from at least one individual post-processed information.

12. A method in accordance with claim 1 further comprising storing the post-processed information within the medical imaging system.

13. A method in accordance with claim 1 further comprising allowing remote access to the medical image information only to authorized users.

14. A method in accordance with claim 1 further comprising limiting the operations available to individuals remotely accessing the medical image information, the limited operations being a sub-set of available operations.

15. A method in accordance with claim 1 further comprising limiting the operations available to individuals remotely accessing the medical image information, the limited operations being a sub-set of available operations and including at least one of controlling movement of a table of the medical imaging system, controlling movement of a gantry of the medical imaging system, controlling movement of the medical imaging system and controlling medical image scanning using the medical imaging system.

16. A method in accordance with claim 1 further comprising tracking at least one of (i) the operations of an individual remotely accessing the medical imaging system and (ii) a duration of time an individual remotely accesses the medical imaging system.

17. A method in accordance with claim 1 wherein the medical imaging system comprises a user interface configured to display medical images from the medical imaging system, and wherein the user interface is remotely accessible.

18. A method in accordance with claim 1 further comprising allowing remote access only after receiving a user authorization input at the medical imaging system.

19. A method in accordance with claim 1 further comprising blocking remote access based on a user input received at the medical imaging system.

20. A method in accordance with claim 1 wherein the performing of post-processing is scheduled to be completed in one of a plurality of predetermined time periods.

21. A method in accordance with claim 1 wherein the medical imaging system comprises a medical imaging device and a workstation and the individuals comprise a group of persons different than the users of the medical imaging device and workstation.

22. A method in accordance with claim 1 wherein the individuals comprise persons associated with an entity different than the entity having the medical imaging system.

23. A method in accordance with claim 1 wherein performing post-processing operations comprises performing post-processing operations after an acquisition of the medical image information and before one of an interpretation and an evaluation of the post-processed medical image information.

24. A method for processing medical image information, said method comprising:
acquiring medical image information using a medical imaging system;
storing the medical image information within the medical imaging system;
accessing the medical image information stored within the medical imaging system from one of a plurality of remote locations;
post-processing the medical image information from one of the plurality of remote locations, the medical image information post-processed using the medical imaging system; and
storing the post-processed medical image information within the medical imaging system.

25. A method in accordance with claim 24 wherein the post-processing is performed by one of a plurality of individuals having expertise in a medical area related to the medical image information.

26. A method in accordance with claim 24 wherein the accessing comprises using virtual private network to access the medical image information.

27. A method in accordance with claim 24 wherein the remote locations are separate physical structures from the structure in which the medical imaging system is located.

28. A method in accordance with claim 24 further comprising configuring the medical imaging system for access via an Internet based interface at each of the remote locations.

29. A method in accordance with claim 24 wherein a plurality of individuals at the remote locations post-process the information, the individuals associated with an entity different than the entity having the medical imaging system.

30. A method for medical diagnostic image processing, said method comprising:
providing a group of individuals for performing post-processing operations on medical image information;
allowing access by the group of individuals to a medical imaging system having stored therein the medical image information, the individuals located remote from the medical imaging system and processing the medical image information using the medical imaging system from the remote location, the individuals associated with an entity different than the entity having the medical imaging system; and
storing the post-processed information within the medical imaging system.

31. A method in accordance with claim 30 wherein the individuals comprise persons having expertise in different areas of medical imaging.

32. A method in accordance with claim 30 wherein the individuals are not associated with an owner of the medical imaging system.

33. A medical imaging system comprising:
a medical imaging device for acquiring medical image information;
a plurality of communication links between the medical imaging device and a plurality of remote locations, the communication links configured for web-based communication; and
a user interface at each of the remote locations for accessing the medical image information within the medical imaging device using the plurality of communication links and for post-processing the medical image information, the post-processing performed by individuals at the remote locations using the medical imaging device.

34. A system for post-processing medical images remotely, said system comprising:
a medical imaging system configured to perform medical imaging operations;
a post-processing system configured to allow performing of post-processing operations remotely; and
a communication link between the medical imaging system and the post-processing system to communicate at least one request for post-processing from the medical imaging system to the post-processing system and configured to provide remote access to the medical imaging system by the post-processing system.

35. A system in accordance with claim 34 further comprising a security layer.

36. A system in accordance with claim 34 wherein the medical imaging system comprises at least one of (i) a medical imaging device and (ii) a workstation.

37. A system in accordance with claim 34 wherein the medical imaging system comprises at least one workstation, the workstation comprising:
a user interface configured to provide co-browsing operations;
a payment and billing component configured to provide online payment operations;
a remote access component configured to control remote access to the medical imaging system;
a tracking component configured to track usage of the medical imaging system; and
a request component configured to generate a post-processing request.

38. A system in accordance with claim 34 wherein the post-processing system comprises:
a post-processing unit configured to remotely access the medical imaging system to perform post-processing operations; and
a data encryption module.

39. A system in accordance with claim 34 wherein the post-processing system comprises:
a remote system viewer configured to provide co-browsing operations; and
an access component configured to provide remote access to the medical imaging system.
